(19) United States
(12) Patent Application Publication
(10) Pub. No.: US 2014/0268058 A1
(43) Pub. Date: Sep. 18, 2014

(54) SYSTEM AND METHOD FOR REMOTELY SUPPLYING PRESCRIPTION EYEGLASSES

(71) Applicant: Clifton Balter, Havertown, PA (US)

(72) Inventor: Clifton Balter, Havertown, PA (US)

(21) Appl. No.: 13/947,036

(22) Filed: Jul. 20, 2013

Related U.S. Application Data
(63) Continuation of application No. 13/843,867, filed on Mar. 15, 2013.

(51) Int. Cl. A61B 3/04

(52) U.S. Cl. CPC A61B 3/04
USPC 351/227

(57) ABSTRACT

Disclosed, in one general aspect, is a sample eyeglass frame that includes a frame having a shape designed for everyday use. The frame includes a left temple having an left earpiece on one end and a left lens interface portion on the other, a right temple having a right earpiece on one end and a right lens interface portion on the other, and a bridge having left and right sides. A left lens interfaces with the left lens interface portion of the left temple and the left side of the bridge, and has a left set of optical measurement markings spanning at least part of the left lens. A right lens interfaces with the right lens interface portion of the right temple and the right side of the bridge, and has a right set of optical measurement markings spanning at least part of the right lens.

### CROSS-REFERENCE TO RELATED APPLICATION

This application is a continuation of application Ser. No. 13/843,867, filed on Mar. 15, 2013, which is herein incorporated by reference.

### FIELD OF THE INVENTION

This invention relates to sample eyeglass frames and kits that can be used to assist in the fitting process, such as when remotely supplying prescription eyeglasses.

### BACKGROUND OF THE INVENTION

A traditional eyewear customer would visit an eyeglass store, choose a frame, and then have an on-site optician take measurements before sending the frames off to have the lenses installed. Since online retailers do not have opticians taking measurements, they have generally relied on the customer to do all of the legwork in order to make a pair of single vision glasses. Bifocals or invisible bifocals (progressives) have generally not been offered online.

In order to accurately fabricate a pair of single vision glasses, there are two measurements needed (in addition to a prescription): the pupillary distance (in millimeters) and the ocular center (in millimeters). To make a pair of bifocals, the necessary measurements are pupillary distance and segment height (in millimeters).

Pupillary distance (PD) is the distance between each pupil and the center of the nose (Monocular PD) or the distance between the pupils (Binocular PD). PD is not relative to a particular pair of glasses. A person's PD is always the same once they reach adulthood, so if a person knows his/her PD, they can report the measurement to an online retailer during the checkout process of buying glasses.

Ocular Center Height (OC Height) is the distance from each pupil to the deepest point of a particular frame (Monocular OC Height), or the average of both pupils to the deepest point (Binocular OC Height). OC Height changes from frame to frame. Generally online retailers ignore the measurement, which leaves the customer with less than perfectly clear vision, and the retailer dealing with remakes and refunds for those who demand lenses to be made as accurately as possible (people have varying degrees of discrimination when it comes to how clearly they expect to see).

Segment Height (Seg Height) is the distance from each pupil to the deepest point of a particular frame (Monocular Seg Height), or the average of both pupils to the deepest point (Binocular Seg Height). Seg Height also changes from frame to frame, and an accurate Seg Height is important to creating a pair of progressive glasses that function properly. OC Height and Segment Height are basically the same measurement but they are called different names because they mean two different things in the fabrication process.

With single vision lenses, the PD and the OC Height are used to align the Optical Center (the clearest point of a single vision lens) directly over the pupil, thus providing the clearest possible vision to the wearer.

When accurately fabricating a pair of bifocals (often invisible bifocals called progressives), the PD and Segment Height measurements are used to line the pupils up properly so that the distance, intermediate and near portions of the lens fall in to the correct place.

When glasses are purchased online, the PD can be retrieved from a customer's eye doctor, or optician that the customer's previous glasses were purchased from. But the hassle customers have to deal with in tracking down their PD can create a situation that discourages sales. Without the OC Height, the glasses will often be less than perfect causing order remakes and customer refunds. And without the Seg Height measurement progressive lenses generally cannot be made.

### SUMMARY OF THE INVENTION

In one general aspect, the invention features a sample eyeglass frame that includes a frame having a shape designed for everyday use. The frame includes a left temple having an left earpiece on one end and a left lens interface portion on the other, a right temple having a right earpiece on one end and a right lens interface portion on the other, and a bridge having left and right sides. A left lens interfaces with the left lens interface portion of the left temple and the left side of the bridge, and has a left set of optical measurement markings spanning at least part of the left lens. A right lens interfaces with the right lens interface portion of the right temple and the right side of the bridge, and has a right set of optical measurement markings spanning at least part of the right lens.

In preferred embodiments the left lens can have a left grid that defines the left set of optical measurement markings and wherein the right lens can have a right grid that defines the right set of optical measurement markings. The left and right lens interface portions can be hinges.

In another general aspect, the invention features a sample eyeglass tryout kit that includes a shipping package. The shipping package holds a plurality of frames of different styles, each having a shape being designed for everyday use. Each of the frames includes a left temple having an left earpiece on one end and a left lens interface portion on the other, a right temple having a right earpiece on one end and a right lens interface portion on the other, a bridge having left and right sides, a left lens that interfaces with the left lens interface portion of the left temple and the left side of the bridge, and has a left set of optical measurement markings spanning at least part of the left lens, and a right lens that interfaces with the right lens interface portion of the right temple and the right side of the bridge, and has a right set of optical measurement markings spanning at least part of the right lens.

In preferred embodiments the left lens can have a left grid that defines the left set of optical measurement markings with the right lens having a right grid that defines the right set of optical measurement markings. The shipping package can further include a return label for the container. The shipping package can further include an instruction sheet that instructs the end user to obtain information about a relationship between the left and right measurement markings and the end user's eyes.

In a further general aspect, the invention features a method of supplying eyeglasses that includes displaying to an end user images of a plurality of eyewear frames of different styles, receiving from the end user a selection of one or more of the displayed frame styles, sending sample frames for each of the selected styles to the end user, receiving from the end user a final selection of one or more of the selected frame styles, and receiving from the end user information measured by the end user about a relationship between the position of the sample frames when worn by the end user and the position of at least one of the end user's eyes for the finally selected frame styles.

In preferred embodiments, the method can further include the steps of initiating manufacture of lenses for the finally selected frame styles based on the information received for the at least one final selection, and conveying the resulting manufactured frames to the end user. The step of receiving can receive either ocular center height information or segment height information. The step of receiving can receive pupillary distance information and either ocular center height information or segment height information. The step of receiving can receive information relative to markings on sample frames that the user has tried on. The step of receiving can receive one or more pairs of grid coordinate values. The step of receiving can receive pointer-based input. The step of receiving pointer-based input can be sensitive to areas smaller than marked areas on the sample frames.

In another general aspect, the invention features an online eyeglass supplying system that includes a frame selection interface operative to allow an end user to select one or more different candidate frame styles for shipment to the end user, a post shipping selection input to receive a final selection of one or more of the selected candidate frame styles, and an input interface for receiving information about a relationship between the position of at least one of the end user's eyes and the position of a candidate frame of a selected style when worn by the end user for the finally selected frame styles.

In preferred embodiments, the input interface can include fields for either ocular center height information or segment height information. The input interface can include fields for pupillary distance information and either ocular center height information or segment height information. The input interface can be responsive to pointer-based input. The input interface can be sensitive to areas smaller than marked areas on the a candidate frame of a selected style.

In another general aspect, the invention features an online eyeglass supplying system that includes means for displaying to an end user images of a plurality of eyewear frames of different styles, means for receiving from the end user a preliminary selection of samples of one or more of the displayed frame styles, means for receiving from the end user a final selection of one or more selected frame styles, and means for receiving from the end user information measured by the end user about a relationship between the position of the sample frames when worn by the end user and the position of at least one of the end user's eyes for the finally selected frame styles.

Systems according to the invention can greatly simplify the work of shopping for eyeglasses remotely. They can also allow for precise specification of bifocals and progressive lenses. And these advantages can be provided relatively inexpensively.

### BRIEF DESCRIPTION OF THE DRAWING

FIG. 1 is a block diagram of an illustrative prescription eyeglass selection system according to the invention,

FIG. 2 is a screenshot of a frame style selection page for the system of FIG. 1,

FIG. 3 is a screenshot of a checkout page for the system of FIG. 1,

FIG. 4 is a screenshot of a prescription navigation page for the system of FIG. 1,

FIG. 5 is a bifocals navigation page for the system of FIG. 1,

FIG. 6 is a schematic diagram illustrating the assembly of a fitting frame tryout kit,

FIG. 7 is a perspective view of a fitting frame for the tryout kit of FIG. 6, and

FIG. 8 is a pupillary position reporting page for the system of FIG. 1.

### DETAILED DESCRIPTION OF AN ILLUSTRATIVE EMBODIMENT

Referring to FIG. 1, a prescription eyeglass selection system according to the invention includes an eyewear selection server 12, which is preferably located at an eyewear supplier location. This server is operatively connected to communicate with a communication network 14 and to interact with a transportation network 16 and optional affiliates 12a. Communication devices 18, such as a computers equipped with Web browsers, are also operative to communicate with communication network from one or more end-user locations 18a . . . 18n. The transportation network is also configured to interact with end users at the end-user locations.

The communication network preferably includes a private or public electronic network, such as the Internet. But other types of communication networks can also be used in different combinations, such as telephone networks, wireless networks, interconnected fax machines, or even physically delivered written communications. The transportation network preferably includes a common carrier such as the U.S. Postal Service, UPS, or Federal Express. But other types of transportation networks could also be used. The illustrative system is used as part of a commercial enterprise where end users shop for and pay for glasses either directly or indirectly through insurance. But aspects of the invention can also be used in other ways, such in part of a corporate vision plan or a charitable eyewear donation program.

In operation, referring to FIGS. 2-3, the eyewear selection server 12 presents HTML webpages to the end-user on his or her browser, although other types of content delivery and user interaction can also be employed. Using product line pages 20 and product pages (not shown), the end user can view one or more frame styles and select them, such as by placing them in an e-commerce shopping cart. The user can then navigate to a checkout page 30 to view and possibly modify his or her selection 32 of frame styles.

Referring to FIG. 4, the user can then move on to a prescription entry page 40. This page provides the end user with several navigation options for prescription lenses, reading glasses and nonprescription lenses. Included in the prescription lenses option is an option for bifocals or progressive lenses. This option will be discussed in more detail, although the functionality described below can be provided for other types of eyeglasses.

Referring to FIGS. 5-6, selecting the option for bifocals leads to a prescription entry sub page, which can include options for mailing fitting frames 72, 72′, 72″ . . . or accessing the end-users previously stored PD, OC height, or segment height measurements for one or more of the selected frame styles. If the user does not have any stored measurements, or chooses not to use them, the user can select the option to have fitting frames sent to his or her location (e.g., home). The eyewear supplier will then prepare a package containing a set of fitting frames for the selected frame styles. The package may also include a pen, fitting instructions, and a return mailing label. Once prepared, the package can be transported to the end user via the transportation network 16.

Referring to FIG. 7, each fitting frame is designed to fit the end user in the same way that the frame style that the user ordered would. To this end, it is preferably a frame of the same brand and model as frame style that the end user ordered. But it could also differ from that frame in ways that do not significantly affect the fit, such by being a different color or being made of a different material.

The fitting frame also includes measurement markings on one or more of the lenses. Preferably, these measurement markings include a rectangular grid 74 on each lens. The grid can be marked with a coordinate system, such as an ABC by 123 coordinate system. Although the rectangular grid is presently preferred, other markings could also be used, such non-rectangular grids or color coded shapes.

In the present embodiment, the markings are printed onto clear non-prescription lenses, although other methods could be used, such as applying stickers to lenses that have a prescription similar to that of the end user. The grid does not need to span the whole lens—it can just occupy an eye area. It is believed that 3 or 4 mm boxes work best. And while it is not presently preferred, the system can work with a single marked lens in at least some situations.

Once the end-user has received the package, he or she will place one of the fitting frames on their face and, if they like the frames, they can have someone look at them to determine which box in the grid is lined up over their pupils. The helper should use an instrument such as a pen, marker, or pencil to mark the spot in the box. To confirm the findings, the shopper can remove and put the glasses back so the helper can check that the markings are in fact over the pupil.

At this point the data is captured and can be conveyed to the optical retailer using a papillary position entry page 80 (see FIG. 8). The user can then enter the positions of his or her pupils relative to the fitting frame markings as grid pairs. This can be done alphanumerically such as by entering coordinate pairs via the computer's keyboard (e.g., C3, D4). The user can also use a widget 82 that will allow the shopper to indicate not only the box on the grid that the pupil resides in, but point and click on a diagram of the lens to show the exact orientation within the box. A third approach is for the end user to upload a digital image of himself with the fitting frames on, and the position information can be extracted from the picture, manually or automatically. Once the papillary positions have been reported for one or more of the selected frame styles, the user can proceed to checkout and confirm his or her final selection.

The supplier now has enough information about the end user's papillary position relative to the finally selected frame styles that to specify lenses to be mounted in the frames. This step can be performed by the supplier or an affiliate fabricator. The finished eyeglasses can then be delivered to the end user.

Although an illustrative browser-based implementation has been presented, systems according to the invention can also be implemented in other ways, such as using special-purpose hardware and/or proprietary software running on one or more general-purpose processors. In addition, while the system was described in connection with a particular set of pages and a specific sequence of steps, these can be varied or supplemented in a number of ways. For example, the different functions could be reached through a different page structure or through different interactive means, such as drop-down menus or voice navigation. Or the system might not include a picture upload option or allow for the selection of more than one type of frame. These countless variations would be within the ability of one of ordinary skill in the art. It is therefore intended that the scope of the present invention be limited only by the scope of the claims appended hereto. In addition, the order of presentation of the claims should not be construed to limit the scope of any particular term in the claims.

What is claimed is:

1. A sample eyeglass frame, comprising:
a frame having a shape designed for everyday use and including:
a left temple having an left earpiece on one end and a left lens interface portion on the other,
a right temple having a right earpiece on one end and a right lens interface portion on the other,
a bridge having left and right sides,
a left lens that interfaces with the left lens interface portion of the left temple and the left side of the bridge, and has a left set of optical measurement markings spanning at least part of the left lens, and
a right lens that interfaces with the right lens interface portion of the right temple and the right side of the bridge, and has a right set of optical measurement markings spanning at least part of the right lens.

2. The apparatus of claim 1 wherein the left lens has a left grid that defines the left set of optical measurement markings and wherein the right lens has a right grid that defines the right set of optical measurement markings.

3. The apparatus of claim 1 wherein the left and right lens interface portions are hinges.

4. A sample eyeglass tryout kit, comprising:
a shipping package, holding
a plurality of frames of different styles, each having a shape being designed for everyday use, and each including:
a left temple having an left earpiece on one end and a left lens interface portion on the other,
a right temple having a right earpiece on one end and a right lens interface portion on the other,
a bridge having left and right sides,
a left lens that interfaces with the left lens interface portion of the left temple and the left side of the bridge, and has a left set of optical measurement markings spanning at least part of the left lens, and
a right lens that interfaces with the right lens interface portion of the right temple and the right side of the bridge, and has a right set of optical measurement markings spanning at least part of the right lens.

5. The apparatus of claim 4 wherein the left lens has a left grid that defines the left set of optical measurement markings and wherein the right lens has a right grid that defines the right set of optical measurement markings.

6. The apparatus of claim 4 wherein the shipping package further includes a return label for the container.

7. The apparatus of claim 4 wherein the shipping package further includes an instruction sheet that instructs the end user to obtain information about a relationship between the left and right measurement markings and the end user's eyes.
